(19) United States
(12) Patent Application Publication
(10) Pub. No.: US 2008/0254078 A1
(43) Pub. Date: Oct. 16, 2008

(54) Chitosan-Based Particles

(76) Inventors: Peter Kauper, Lausanne (CH); Carsten Laue, Nyon (CH)

Correspondence Address: STURM & FIX LLP, 206 SIXTH AVENUE, SUITE 1213, DES MOINES, IA, 50309-4076, US

(21) Appl. No.: 12/066,885

(22) PCT Filed: Sep. 16, 2005
(86) PCT No.: PCT/IB05/02744

§ 371 Date: Mar. 14, 2008

(51) Int. Cl. A61K 9/14; A61K 8/02; A61K 47/36; A61K 38/02; A61K 31/70

(52) U.S. Cl. 424/401; 424/499; 514/777; 514/2; 514/44

(57) ABSTRACT

The present invention provides a new binary system of hydrophilic nanoparticles and microparticles, using chitosan and polyanionic polysaccharides carrying carboxymethyl groups, sulfate groups or carboxy plus sulfate groups.

### BACKGROUND OF THE INVENTION

1. Field of the Invention

The invention relates to the fields of polymer chemistry, colloid chemistry, polyelectrolyte chemistry, biomedical engineering, pharmaceutical sciences, cosmetic engineering and food industry. More specifically, the present invention relates to a novel nanoparticle system.

2. Description of the Prior Art

Nanosized systems are submicroscopic systems defined by sizes below 1 micrometer. Nanoparticles are submicroscopic particles. Systems above 1 micrometer in size are named microparticulate. Both, microparticles as well as nanoparticles are used as carrier systems e.g. for drugs, pro drugs, proteins and peptides, enzymes, vitamins, fragrances, etc. In such systems, microparticles and nanoparticles are formed in a mixture with the molecules of interest to be encapsulated within the particles for subsequent sustained release.

Hydrophilic microparticles or nanoparticles can be produced in various ways. One option is to process the hydrophilic materials inside oil droplets of a water-in-oil emulsion, but this route implies to use substances such as organic solvents and detergents which are often not tolerated by complex biological materials or systems. A more promising way to produce hydrophilic particles relies on the interactive forces between oppositely charged polyanions and polycations, a process which can be run under mild conditions which are not detrimental to complex biological materials or systems. This route is characterized by the absence of organic solvents, of detergents, of acidic or alkaline pH values. Salts in physiological quantities may be present during particle formation. Many hundreds of combinations of polyanions and polycations were examined for polyelectrolyte complex formation in hydrophilic microparticulate and nanoparticulate systems. Only a few combinations were found to be usable.

A drawback of the known binary encapsulation systems (i.e. one polyanion and one polycation) is instability towards dilution or salts, i.e. limited stability in physiological environment. As a consequence, with time the nanoparticles or microparticles disintegrate or tend to form aggregates of sizes above several hundreds of micrometers.

To overcome the limitations of such binary systems, ternary (i.e. three ionic components) and quaternary systems (i.e. four ionic components) comprising polyelectrolytes and electrolytes of low molar mass or salts were proposed for microparticulate as well as for nanoparticulate systems (A. Prokop, U.S. Pat. No. 6,482,439, U.S. Pat. No. 6,726,934 and US patent application 20030170313). An example is pregelation of the polysaccharide alginate with calcium cations prior to nanoparticle formation with a further polysaccharide, chitosan (S. De and D. Robinson, Polymer relationships during preparation of chitosan-alginate and poly-l-lysine-alginate nanospheres, J. Controlled Release, 89 (2003) 101-112; K. L. Douglas and M. Tabrizian, Effect of experimental parameters on the formation of alginate-chitosan nanoparticles and evaluation of their potential application as DNS carrier, J. Biomater. Sci. Polymer Edn., 16 (2005) 43-56). However, such ternary and quaternary systems imply additional ingredients, involve more preparation steps than binary systems, and are often complicated to produce.

Prior art includes only a limited number of binary systems designed to create hydrophilic nanoparticles by ionic complex formation. One can mention the system “chitosan plus tripolyphosphate”, “chitosan plus alginate” and “poly-L-lysine plus alginate”.

The system “chitosan plus tripolyphosphate (TPP)” is based on the interaction between a polymer (chitosan, a polycation at pH values below approximately 6) and an oligomer, the trimeric phosphate tripolyphosphate (M. J. Alonso Fernandez et al., U.S. Pat. No. 6,649,192; A. Vila et al., Low molecular weight chitosan nanoparticles as new carriers for nasal vaccine delivery in mice, Europ. J. Pharm. and Biopharm., 57 (2004) 123-131; K. A. Janes et al., Polysaccharide colloidal particles as delivery systems for macromolecules, Adv. Drug Del. Rev., 47 (2001) 83-97; L. Qi and Z. Xu, Lead sorption from aqueous solutions on chitosan nanoparticles, Colloids and Surfaces A: Physiochem. Eng. Aspects 251 (2004) 183-190; P. Calvo et al., Novel Hydrophilic Chitosan-Polyethylene Oxide Nanoparticles as Protein Carriers, Journal of Applied Polymer Science 63 (1997) 125-132; Y. Wu et al., Chitosan nanoparticles as a novel delivery system for ammonium glycyrrhizinate, Int. J. Pharm., 295 (2005) 235-245; L. Chena and M. Subirade, Chitosan/beta-lactoglobulin core-shell nanoparticles as neutraceutical carriers, Biomaterials, 26 (2005) 6041-6053; Y. Aktas et al., Preparation and in vitro evaluation of chitosan nanoparticles containing a caspase inhibitor, International Journal of Pharmaceutics 298 (2005) 378-383). We note that the “chitosan-TPP” particles described in the above-mentioned literature have invariably a positive surface charge, expressed by positive zeta potentials.

“Chitosan plus alginate” nanoparticles are briefly described in the work focusing on the ternary system “calcium cation plus alginate plus chitosan”. This system tends to form, beside the colloidal nanoparticles, macroscopic aggregates (S. De and D. Robinson, Polymer relationships during preparation of chitosan-alginate and poly-l-lysine-alginate nanospheres, J. Controlled Release, 89 (2003) 101-112; K. L. Douglas and M. Tabrizian, Effect of experimental parameters on the formation of alginate-chitosan nanoparticles and evaluation of their potential application as DNS carrier, J. Biomater. Sci. Polymer Edn., 16 (2005) 43-56).

“Alginate plus PLL” nanoparticles are described in the work focusing on quaternary systems. However, the untreated particles showed instability when washed with water or saline (A. Prokop, US patent application 20030170313).

Two recently published review articles underline the interest towards the polycation chitosan for biomedical applications, particularly in polyelectrolyte complexes between a polyanion and chitosan. One article focuses on the release systems as well as on the biomedical application of the chitosan complex (J. Berger, M. Reist, J. M. Mayer, O. Felt, N. A. Peppas, R. Gurny, Structure and interactions in covalently and ionically crosslinked chitosan hydrogels for biomedical applications, Eur. J. Pharm. Biopharm., 57 (2004) 19-34). The second review article provides a detailed outline of the interactions between chitosan and different polyanions such as anionic polysaccharides, proteins or synthetic polymers: e.g. the nature of the complexing anionic group (carboxy, sulfate and phosphate groups) as well as the nature of the complex type (precipitate, macroscopic hydrogel, droplets in the millimeter range, microparticles) (J. Berger, M. Reist, J. M. Mayer, O. Felt, R. Gurny, Structure and interactions in chitosan hydrogels formed by complexation or aggregation for biomedical applications, Eur. J. Pharm. Biopharm., 57 (2004) 35-52).

Chitosan-based formulations are generally considered appropriate for biomedical applications due to the so-called absorption-enhancing effect of chitosan, i.e. the opening of the intercellular tight junctions favoring the paracellular drug transport (I. M. van der Lubben, J. C. Verhoef, G. Borchard, H. E. Junginger, Chitosan and its derivatives in mucosal drug and vaccine delivery, Eur. J. Pharm. Sci., 14 (2001) 201-207).

Hydrophilic particles based on chitosan are of growing interest, as witnessed by the growing amount of literature in the field. A recent paper reviews the use of chitosan in micro- and nanoparticles in drug delivery (review article S. A. Agnihotri, et al., Recent advances on chitosan-based micro- and nanoparticles in drug delivery, Journal of Controlled Release 100 (2004) 5-28). The use of the prior art particles as delivery means for bioactive molecules such as proteins, peptides, antigens, oligonucleotides, RNA and DNA fragments, growth factors, hormones or other bioactive molecules is nevertheless limited, because the preparation of the particles requires physical or chemical interventions which are susceptible to destroy or inactivate such bioactive molecules (review paper U. Bilati, E. Allémann, E. Doelker, Strategic approaches for overcoming peptide and protein instability within biodegradable nano- and microparticles, Eur. J. Pharm. Biopharm., 59 (2005) 375-388). Destruction or inactivation of bioactive molecules occurs due to organic solvents, preparation processes involving emulsification, aldehydic crosslinking, acidic or alkaline preparation conditions.

Chitosan is a natural polymer composed of glucosamine units. It is produced out of crustacean shells or out of biotechnological processes. Chitosan is nearly exclusively derived from chitin by a deacetylation process of chitin. Chitin is a beta-1,4-linked N-acetylglucosamine whereas chitosan is the corresponding beta-1,4-glucosamine. Neither chitin nor chitosan are homopolymers as both contain varying fractions of the acetyl moieties on the glucosamine repeating unit. They can be distinguished by their solubility in aqueous acidic conditions. At degrees of acetylation of approximately higher than 40% the N-acetylglucosamine is insoluble and is named chitin whereas the soluble N-acetylglucosamine is named chitosan.

Chitosan is available in the market in a variety of forms. Chitosan samples differ in molar mass and in the degree of deacetylation. Furthermore, chitosan is available in the form of different salts. Chitosan is known for its excellent biocompatibility, and is therefore part of many pharmaceuticals formulations (S. Hirano, H. Seino, Y. Akiyama, I. Nonaka, Chitosan: A biocompatible material for oral and intravenous administrations. In: Gebelein G G and Dunn R L, eds. Progress in biomedical polymers. New York: Plenum Press (1990) 283-289). Chitosan is insoluble in aqueous solutions of neutral pH values, and soluble at slightly acidic pH values. As the molar mass decreases below approximately 10000 g/mol, chitosan becomes soluble at close to neutral to neutral pH values. Such soluble at neutral pH value chitosans are sometimes named oligochitosan (S. Y. Chae, M.-K. Jang, J.-W. Nah, Influence of molecular weight on oral absorption of water soluble chitosans, Journal of Controlled Release, 102 (2005) 383-394).

Accordingly, there is a need for a simple biocompatible chitosan-based binary particulate system. More specifically, there is a need for a chitosan-based binary system and a production process which can yield particles not only with a positive surface charge, but a system which can be operated to also produce particles with a negative surface charge. The particle production should be possible under simple conditions and reproducibly resulting in a stable nanoparticle or microparticle system. Defined behavior towards physiological environments including elevated temperatures is a prerequisite for applications in medical, pharmaceutical, biotechnological and other fields. Such defined behaviors can be long term stability of particles, controlled enzymatic degradation of particles or triggered particle disintegration.

### BRIEF DESCRIPTION OF THE INVENTION

For the purpose of this invention (specification including claims), the term “polysaccharide” shall mean a saccharide having at least two sugar units.

The present invention is directed to hydrophilic particles consisting of chitosan and one type of anionic polysaccharide, said polysaccharide being selected from the group consisting of polysaccharides carrying carboxymethyl moieties, polysaccharides carrying sulfate moieties, polysaccharides carrying sulfate and carboxy moieties, and hyaluronic acid.

In a preferred embodiment of the invention, said particles are characterized in that said polysaccharide is selected from the group of carboxymethyl dextran, carboxymethyl cellulose, carboxymethyl amylose, carboxymethyl beta-cyclodextrin, dextran sulfate, cellulose sulfate, chondroitin sulfate, heparin, heparan sulfate, dermatan sulfate and keratan sulfate, and in that the weight ratio between said polysaccharide and chitosan in the respective solutions is within a range of 3:1 to 1:10.

The particles according to the invention may additionally be characterized in that a moiety, a biologically functional group or a prodrug is covalently bound to the chitosan, to said anionic polysaccharide, or to both.

In a further embodiment of the invention, the particles according to the invention may further comprise one or more uncharged polymers. Said uncharged polymer(s) may be any uncharged polymer, or be selected from the group consisting of polyethylene glycol, polyethylene glycol derivatives, polysaccharides and polysaccharide derivatives.

In a further embodiment, the particles according to the invention may additionally comprise one or more multivalent cation(s) selected from the group consisting of calcium, barium, strontium, aluminium and iron.

In still a further embodiment of the invention, the particles according to the invention may additionally comprise one or more biologically active substance or substances. Said biologically active substance or substances may be any biologically active substance, or be selected from the group consisting of pharmaceuticals, prodrugs, proteins, DNA, RNA, hormones, vitamins, cosmetics, fragrances and flavors.

The particles according to the invention may be microparticles or nanoparticles.

The present invention also provides a composition selected from the group of a pharmaceutical composition, a cosmetic composition, a food composition or a dermo-pharmaceutical composition, comprising an effective amount of particles according to the invention.

The present invention also provides the use of the particles according to the invention for the transport and the concentration of biologically active substances in a biological system.

The present invention further provides a process for making hydrophilic particles consisting of chitosan and one type of anionic polysaccharide, said polysaccharide being selected from the group consisting of polysaccharides carrying carboxymethyl moieties, polysaccharides carrying sulfate moieties, polysaccharides carrying sulfate and carboxy moieties, and hyaluronic acid, comprising:
i. preparing an aqueous solution of said anionic polysaccharide.
ii. preparing an aqueous solution of chitosan.
iii. slowly adding the solution obtained in (i) or (ii) to the other solution.

Preferably, the process according to the invention is characterized in that said polysaccharide is selected from the group of carboxymethyl dextran, carboxymethyl cellulose, carboxymethyl amylose, carboxymethyl beta-cyclodextrin, dextran sulfate, cellulose sulfate, chondroitin sulfate, heparin, heparan sulfate, dermatan sulfate, keratan sulfate and hyaluronic acid, and in that the weight ratio between said oligosaccharide or polysaccharide and chitosan in the respective solutions is within a range of 3:1 to 1:10.

Additionally, the process according to the invention may be characterized in that one or more of the following additional components are present in at least one of said aqueous solutions:
one or more uncharged polymer(s) selected from the group consisting of polyethylene glycol, polyethylene glycol derivatives, polysaccharides and polysaccharide derivatives.
one or more multivalent cation(s) selected from the group consisting of calcium, barium, strontium, aluminium and iron.
one or more biologically active substance(s)

The process according to the invention may further comprise the step of incorporating or coating one or more biologically active substance(s) into or onto said particles after formation of said particles.

The process according to the invention may also further comprise the step of incorporating or coating polyanions or polycations into or onto said particles after formation of said particles.

The process according to the invention may also further comprise the step of covalently crosslinking particles after formation of said particles.

### DESCRIPTION OF THE FIGURE

FIG. 1 shows infrared spectroscopy data of nanoparticles from example 1. From top to bottom: spectrum for nanoparticles from example 1, for chitosan, and for chondroitin sulfate.

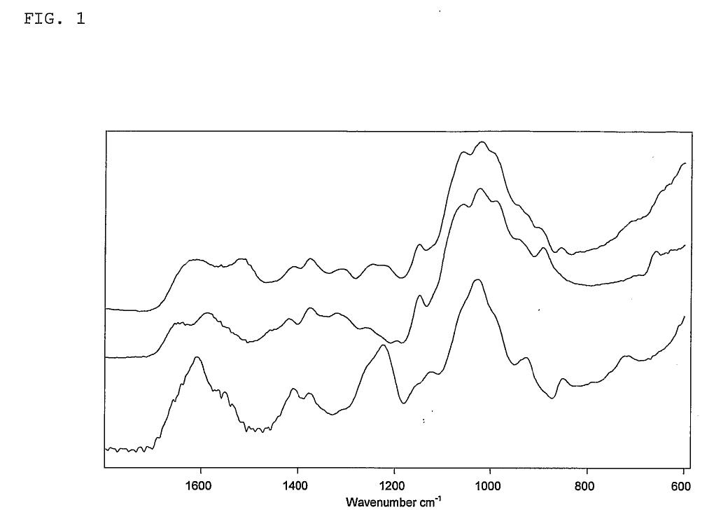

### DESCRIPTION OF THE INVENTION

The particles according to the invention are constituted of only two hydrophilic polymers, one of which exhibits a negative charge (polysaccharide type polyanion or oligoanion), and chitosan exhibiting a positive charge.

Polysaccharides may be divided into classes characterized by the nature of their anionic group. One class of anionic polysaccharides is constituted by polysaccharides carrying carboxymethyl groups. Examples for this class are carboxymethyl dextran, carboxymethyl cellulose, carboxymethyl amylose, carboxymethyl beta cyclodextrin. A further class is constituted by polysaccharides carrying sulfate groups. Examples for this class are dextran sulfate and cellulose sulfate. A yet further class carries more than one type of anionic group. Glucosamineglucans (GAGs), straight chain acidic polysaccharides, generally carry carboxy groups and additionally sulfate groups. Examples are chondroitin sulfate and heparin. Despite belonging to the class of glucosamineglucans, the polysaccharide hyaluronic acid carries no sulfate groups but only carboxy groups.

The above mentioned classes of polysaccharides serve as examples for the general concept that carboxymethyl group carrying, sulfate group carrying, or carboxy and sulfate group carrying polysaccharides form nanoparticles and microparticles under specific conditions.

Carboxymethyl cellulose, cellulose sulfate, dextran sulfate, carboxymethyl dextran and carboxymethyl amylose are polysaccharides obtained by chemical reaction respectively from the natural polymers cellulose, dextran and amylose. Depending on the reaction, the carboxymethyl or sulfate group can be introduced in different amounts, in different positions, and/or with different distributions along the chain. The product is available in different degrees of carboxymethylation and sulfation and molar masses, in form of the sodium salt or of other salts. Sodium carboxymethyl-beta-cyclodextrin is one of the common anionic derivatives of a cyclic saccharide, the cyclodextrin. Cyclodextrins are composed of several anhydroglucose units (most common are 6, 7 or 8 membered rings, and respectively named alpha, beta or gamma cyclodextrin). Sulfated or carboxymethylated derivates represent some anionic derivatives of the cyclodextrins.

The term glucosaminoglycans (GAGs) describes a family of polysaccharides carrying carboxy groups and additionally sulfate groups. GAGs commonly found in mammals include hyaluronic acid, chondroitin-4 sulfate, chondroitin-6 sulfate, dermatan sulfate, keratin sulfate, heparan sulfate and heparin. Within this group of anionic polymers, hyaluronic acid is not carrying a sulfate group. All other polysaccharides carry carboxy groups as well as sulfate groups in different amounts. Generally, GAGs exhibit low molar masses (in the order of 4,000-50,000 g/mol) with exception of hyaluronic acid (up to 10,000,000 g/mol). Nevertheless, by degradation, hyaluronic acid or its salts can reach lower molar masses of any desired value. Synthetic polysaccharides of very low molar mass (of approx. 1,000-5,000 g/mol) can exhibit heparin functionality (anti-coagulant). Similarly to heparin, these synthetic polysaccharides carry sulfate and carboxy groups.

The overall electrical surface charge of the particles according to the invention, measurable as zeta potentials, can vary depending on the ratio of the two hydrophilic polymers. Surprisingly, zeta potentials in a very broad range (from highly positive values to highly negative values) can be produced by changing production parameters. This is in contrast with prior art nanoparticles from chitosan plus tripolyphosphate, which are limited to positive zeta potentials (M. J. Alonso Fernandez et al., U.S. Pat. No. 6,649,192). After preparation of the nanoparticles according to the invention, the resulting zeta potential may be adjusted by adding additional ingredients charged oppositely to the particle surface charge.

The size of the micro- and/or nanoparticles according to the invention can be modulated as well, from a few nanometers to a few micrometers, by adequately selecting the preparation conditions such as selection of polyanion, concentration of polyanion and polycation, presence and concentration of salts and presence, nature and concentration of uncharged polymers. The size of the micro- and/or nanoparticles can also be selected after completion of the particle preparation procedure, by filtration and/or dialysis techniques.

Another aspect of the present invention is the possibility to covalently link a moiety or functionality to one of the polymer compounds of the micro- and/or nanoparticles prior to particle formation. For applications in the food, cosmetics or pharmaceutical industry or in medicine, such moieties or functionalities might target for example a receptor interaction. Furthermore, a drug or pro drug can be covalently linked to one of the polymer compounds of the particle prior to the micro- and/or nanoparticle formation.

Surprisingly, the formation of the micro- and/or nanoparticles of the present invention occurs spontaneously by a colloid formation of the binary system polysaccharide and chitosan. The formation of the nanoparticles can be directly detected by the human eye by the so-called “Tyndall effect”. This term shall refer to light diffusion in many directions by large molecules and small particles resulting in slightly milky solutions. The solvent system for the both components of the particles according to the invention can vary from water to salt solutions, and can cover a wide range of pH values depending on chitosan type, including physiological pH values. To a certain degree, water miscible solvents can be present. This process can also be considered as ionic gelation, ionic crosslinking, coacervation or polyelectrolyte complex formation of the two components. The polyelectrolyte complex formation process is extensively described in literature.

This invention also provides a simple process of microparticle and nanoparticle preparation, by simply dropping one component in an aqueous solution into another aqueous solution containing the second compound of opposite charge under mild mechanical mixing of the two components. No special attention has to be paid to the size of the droplets, or the flow rate of the solution of the first component dropped into the second solution. Prior art inventions use techniques in which a nanoscale mist of droplets must be produced, either by a hollow ultrasound probe (A. Prokop, U.S. Pat. No. 6,726,934 and US patent application 20030170313) or by double nozzle atomizer (US patent application 20040136961), or by direct ultrasonication (S. De, D. Robinson, Polymer relationships during preparation of chitosan-alginate and poly-l-lysine-alginate nanospheres, J. Controlled Release, 89 (2003) 101-112).

Nanoparticle formation is affected by the amount (relative proportion) of anionic compound dropped into the solution of the cationic compound. As a result of these proportions, the micro- and/or nanoparticles of the present invention might be composed in high excess by the cationic compound (chitosan) or might be composed in high excess by the anionic compound (anionic polysaccharide). Accordingly, the micro- and/or nanoparticles have a high positive zeta potential or a high negative zeta potential respectively. This is reflected by the surface charges of the micro- and/or nanoparticles up to +63 mV or up to −50 mV respectively. For example, when the weight ratio of the polyanion heparin to the polycation chitosan is approximately 3:1, stable nanoparticles result with negative zeta potential. Whereas, when the weight ration of the polycation chitosan to the polyanion heparin is approximately 2:1, stable nanoparticles with positive zeta potential result.

In addition to the polyanion and the chitosan, other components may be added during the micro- and/or nanoparticle formation. Examples are multivalent cations such as calcium, uncharged polymers such as polyethylene glycol, or uncharged saccharide derivatives.

In view of their further use, micro- and nanoparticles formulations according to the present invention may undergo solvent changes, purification (e.g. by dialysis), wet heat sterilization, may be dried by freeze drying and spray drying, among other techniques, etc.

The incorporation or coating of charged molecules of interest within or on the micro- or nanoparticles of the present invention (loading) can be achieved by a simple and mild procedure of ionic interaction between the positively or negatively charged micro- or nanoparticle, and a negatively, or partially negatively, or a positively, or partially positively, respectively charged molecule or an uncharged molecule linked, covalently or by other means, to a moiety carrying negative charges. The incorporation or coating with negatively or positively charged molecules will evidently direct the zeta potential of the resulting micro- or nanoparticle closer to neutral values.

The association of bioactive molecules may also comprise mechanisms of physical entrapment. Bioactive molecules of high molar mass or molecules of low molar mass covalently bond to uncharged polymers can be present during micro- or nanoparticle formation, and consequently associated by a physical entrapment process.

The micro- or nanoparticles of this invention are presented as colloidal suspensions in an aqueous medium in which other ingredients could eventually be incorporated, not or partially interacting with the micro- or nanoparticles: organic solvents, salts, acids, bases, cryoprotectives, detergents, preservatives, viscosity enhancers.

One targeted application for the micro- and nanoparticles of the present invention is the delivery and the transport within the human or animal body of bioactive molecules, mainly bioactive macromolecules such as biologically active polysaccharides, proteins, peptides, antigens, oligonucleotides, RNA and DNA fragments, growth factors, hormones etc. Another important targeted application is the delivery within the human or animal body of small organic molecules such as pharmaceuticals. Additional applications comprise but are not limited to immobilization of biologic or synthetic molecules for food applications, flavor delivery and fragrance delivery applications.

With respect to the administration routes of micro- and nanoparticles to the human or animal body, the modulation of the zeta potential of the nanoparticles is of importance. Generally, epithelial and mucosal routes, due to the negatively charged surface of the epithelium or mucosa, favor the application of positively charged micro- or nanoparticles. Whereas the parenteral routes, especially intravenous administration, generally favor the application of neutral to only slightly positively or negatively charged micro- or nanoparticles.

The particles of the present invention offer several advantages over other types of micro- or nanoparticles described in the prior art. Their preparation is very simple, as it does not require complicated droplet atomization techniques such as ultrasonication techniques. It does not require any potentially harmful ingredients and solvents, such as are organic solvents, oils and aldehydic crosslinking agents for incorporating the bioactive molecule of interest in the nanoparticle. The particles according to the invention can achieve an unprecedented range of zeta potentials, ranging from highly positive to highly negative zeta potentials. The incorporation of bioactive molecules into the nanoparticles of the present invention is carried out with great flexibility and under a multitude of conditions: broad range of pH values and different salt concentrations. Finally, the physicochemical properties of the micro- or nanoparticles such as their surface charge or their size can be modulated by simple means.

### EXAMPLES

### Example 1

420 ml of a solution of 0.1% chondroitin sulfate (type A, Sigma Chemicals) in water at pH7 were added to a solution of 70 ml chitosan (high viscosity type) of 0.025% in aqueous HCl at pH4.6. Addition was slow, by dropping into a stirred solution. Opalescence appeared after the first added droplets and became more and more intense. The final dispersion was filtered with a 1.2 μm filter and then dialyzed against water by means of a 0.2 μm membrane. A milky, opalescent dispersion with visible Tyndall effect resulted which remain unchanged after filtration through a 0.8μ. By filtration through a 0.45 μm filter the intensity of the Tyndall effect was reduced. By filtration through a 0.22 μm filter a clear solution was obtained as filtrate. The zeta potential was measured at −50 mV.

### Example 2

5 ml of a solution of 0.1% dextran sulfate (from Leuconostoc spp., Sigma Chemicals) in water at pH7 were added to a solution of 20 ml oligochitosan (Mₙ 4500 g/mol, M_{w} 6000 g/mol) of 0.025% in aqueous HCl at pH5.5. Addition was slow, by dropping into a stirred solution. Opalescence appeared after the first added droplets and became more and more intense. The milky, opalescent dispersion with visible Tyndall effect resulted which remain unchanged after filtration through a 1.2 μm and 0.8 μm filter. By filtration through a 0.45 μm filter the intensity of the Tyndall effect was reduced. By filtration through a 0.22 μm filter a clear solution was obtained as filtrate.

### Example 3

20 ml of a solution of 0.1% heparin (from porcine intestinal mucosa, Fluka Biochemika) in water at pH7 were added to a solution of 150 ml chitosan (middle viscosity type) of 0.025% in aqueous HCl at pH4.6. Addition was slow, by dropping into a stirred solution. Opalescence appeared after the first added droplets, and became more and more intense. The final dispersion was dialyzed against water and then concentrated to a final volume of 80 ml by means of a 0.2 μm membrane. A milky, opalescent dispersion with visible Tyndall effect resulted. By filtration through a 1.2 μm filter or through a 0.8 μm filter the intensity of the Tyndall effect was very slightly reduced. By filtration through a 0.45 μm filter a clear solution was obtained as filtrate. The zeta potential was measured at +63 mV.

### Example 4

200 ml of a solution of 0.5% carboxymethyl amylose (Sigma Chemicals) in water at pH7, were added to a solution of 300 ml chitosan (middle viscosity type) of 0.1% in aqueous HCl at pH5.5. Addition was slow, dropping into a stirred solution. Opalescence appeared after the first added droplets, and became more and more intense. The final dispersion was dialyzed against water by means of a 0.2 μm membrane. A milky, opalescent dispersion with visible Tyndall effect resulted which remain unchanged after filtration through a 0.8 μm filter. By filtration through a 0.45 μm filter the intensity of the Tyndall effect was reduced. By filtration through a 0.22 μm filter a clear solution was obtained as filtrate. The zeta potential was measured at +50 mV.

1. Hydrophilic nanoparticles consisting of chitosan and one type of anionic polysaccharide, said polysaccharide being selected from the group consisting of carboxymethyl dextran, carboxymethyl amylose, carboxymethyl beta-cyclodextrin, dextran sulfate, cellulose sulfate, chondroitin, sulfate, heparin, heparan sulfate, dermatan sulfate, keratan sulfate and hyaluronic acid, obtainable by slowly adding one of the chitosan and polysaccharide in one aqueous solution to another aqueous solution containing the other of the chitosan and polysaccharide and chitosan in the respective solutions is within a range of 3:1 to 1:10.

2. Nanoparticles of claim 1, in the form of a colloidal suspension in an aqueous medium in which other ingredients can be incorporated.

3. Nanoparticles of claim 1 modified in that a moiety, a biological functional group or a prodrug is covalently bound to the chitosan, to said anionic polysaccharide, or to both.

4. Nanoparticles of claim 1, modified by further comprising one or more uncharged polymers.

5. Nanoparticles of claim 4, modified in that said uncharged polymer or polymers is/are selected from the group consisting of polyethylene glycol, polyethylene glycol derivatives, polysaccharides and polysaccharide derivatives.

6. Nanoparticles of claim 1, modified by further comprising one or more multivalent cation(s) selected from the group consisting of calcium, barium, strontium, aluminium and iron.

7. Nanoparticles of claim 1, modified in that said particles further comprise one or more biologically active substance or substances.

8. Nanoparticles of claim 7, modified in that said biologically active substance or substances is/are selected from the group consisting of pharmaceuticals, prodrugs, proteins, DNA, RNA, hormones, vitamins, cosmetics, fragrances and flavors.

9-10. (canceled)

11. A composition selected from the group of a pharmaceutical composition, a cosmetic composition, a food composition or a dermo-pharmaceutical composition, comprising an effective amount of nanoparticles as defined in claim 1.

12. Use of the particles of claim 1 for the transport and the concentration of biologically active substances in a biological system.

13. A process for making hydrophilic nanoparticles consisting of chitosan and one type of anionic polysaccharide, said polysaccharide being selected from the group consisting of carboxymethyl dextran, carboxymethyl amylose, carboxymethyl beta-cyclodextrin, dextran sulfate, cellulose sulfate, chondroitin sulfate, heparin, heparin sulfate, dermatan sulfate, keratan sulfate and hyaluronic acid, comprising:
a. preparing an aqueous solution of said anionic polysaccharide.
b. preparing an aqueous solution of chitosan.
c. slowly adding the solution obtained in (i) or (ii) to the other solution, wherein the weight ratio between said polysaccharides and chitosan in the respective solutions is within a range of 3:1 to 1:10.

14. The process of claim 13, wherein one aqueous solution is slowly added to the other aqueous solution by dropping.

15. The process of claim 13, modified in that one or more of the following additional components are present in at least one of said aqueous solutions:
one or more uncharged polymer(s) selected from the group consisting of polyethylene glycol, polyethylene glycol derivatives, polysaccharides and polysaccharide derivatives.
one or more multivalent cation(s) selected from the group consisting of calcium, barium, strontium, aluminium and iron.
one or more biologically active substance(s).

16. The process of claim 13, modified by further comprising the step of incorporating or coating one or more biologically active substance(s) into or onto said nanoparticles after formation of said nanoparticles.

17. The process of claim 13, modified by further comprising the stop of incorporating or coating polyanions or polycations into or onto said nanoparticles after formation of said nanoparticles.

18. The process of claim 13, further comprising the step of covalently crosslinking nanoparticles after formation of said nanoparticles.
